(19) United States
(12) Patent Application Publication
(10) Pub. No.: US 2014/0056868 A1
(43) Pub. Date: Feb. 27, 2014

(54) Supercoiled MiniVectors as a Tool for DNA Repair, Alteration and Replacement

(71) Applicants: Lynn E. Zechiedrich, Houston, TX (US); Jonathan Fogg, Houston, TX (US); Daniel James Catanese, JR., Houston, TX (US); Erol Bakkalbasi, Houston, TX (US); Nancy Maizel, Seattle, WA (US); Olivier Humbert, Seattle, WA (US)

(72) Inventors: Lynn E. Zechiedrich, Houston, TX (US); Jonathan Fogg, Houston, TX (US); Daniel James Catanese, JR., Houston, TX (US); Erol Bakkalbasi, Houston, TX (US); Nancy Maizel, Seattle, WA (US); Olivier Humbert, Seattle, WA (US)

(73) Assignees: University of Washington Center for Commercialization, Seattle, WA (US); Baylor College of Medicine, Houston, TX (US)

(21) Appl. No.: 13/906,130

(22) Filed: May 30, 2013

Related U.S. Application Data
(60) Provisional application No. 61/653,279, filed on May 30, 2012.

(51) Int. Cl. C12N 15/85

(52) U.S. Cl. CPC C12N 15/85
USPC 424/94.6; 435/320.1; 435/196; 435/325; 435/419; 435/243; 435/462; 514/44 R

(57) ABSTRACT

In some embodiments the present disclosure provides a composition for targeted alteration of a DNA sequence and methods of altering the targeted DNA sequence using the composition. In some embodiments such a composition comprises a MiniVector comprising a nucleic acid sequence template for homology-directed repair, alteration, or replacement of the targeted DNA sequence within a cell in vivo or in vitro, where the MiniVector lacks both a bacterial origin of replication and an antibiotic selection gene, and wherein the MiniVector has a size up to about 2,500 base pairs. In further embodiments of the present disclosure the nucleic acid sequence template for the homology-directed repair, alteration, or replacement of the targeted DNA sequence comprises at least one portion of the template complementary to a nucleic acid sequence near the targeted DNA sequence to be altered; and at least one portion of the template which is not complementary to the targeted DNA sequence to be altered, wherein the non-complementary portion of the nucleic acid template contains the alteration desired in the targeted DNA sequence. In some embodiments, the composition further comprises at least one site-specific nuclease. Further embodiments of the present disclosure pertain to a method of treating a genetic disorder, or other condition, in a subject in need thereof.

### CROSS-REFERENCE TO RELATED APPLICATIONS

This application claims priority to U.S. Provisional Application No. 61/653,279 filed on May 30, 2012. The entirety of the aforementioned application is incorporated herein by reference.

### STATEMENT REGARDING FEDERALLY SPONSORED RESEARCH

The invention was supported, in whole or in part, by grant numbers R01-AI054830 and RL1-GM084434 from the National Institutes of Health. The Government has certain rights in the invention.

### FIELD

This invention relates to compositions and methods of gene therapy using MiniVectors™ comprising a nucleic acid sequence as a tool for DNA repair, alteration, or replacement.

### BACKGROUND

Targeted genome engineering involves editing or altering endogenous DNA in a directed manner at a specific site along the DNA within the cell. Despite the tremendous potential of gene repair and homology-directed gene alteration, current genome engineering approaches provide very low efficiency of repair or editing and have the potential to introduce harmful or undesired DNA sequences and outcomes. Therefore, there is a need to develop more effective methods of targeted genome engineering, that are stable in biological environments and that allow for greater cell transfection and transgene expression.

### SUMMARY

In some embodiments the present disclosure provides a composition for alteration of a targeted DNA sequence. For example, in a non-limiting embodiment such a composition comprises a MiniVector comprising a nucleic acid sequence template for homology-directed repair, alteration, or replacement of the targeted DNA sequence within a cell in vivo or in vitro, where the MiniVector lacks both a bacterial origin of replication and an antibiotic selection gene, and where the MiniVector has a size up to about 2,500 base pairs. In further embodiments of the present disclosure the nucleic acid sequence template for the homology-directed repair, alteration, or replacement of the targeted DNA sequence comprises at least one portion of the template complementary to a nucleic acid sequence near the targeted DNA sequence; and at least one portion of the template which is not complementary to the targeted DNA sequence, where the non-complementary portion of the nucleic acid template contains the alteration desired in the targeted DNA sequence. In some embodiments, the composition further comprises at least one site-specific nuclease.

In another embodiment, the present disclosure relates to a method of altering a target DNA sequence in a cell. In some embodiments such a method comprises transfecting a MiniVector comprising a nucleic acid sequence template. In some embodiments the nucleic acid sequence template comprises at least one portion complementary to a nucleic acid sequence near the target DNA sequence; and at least one portion which is not complementary to the target DNA sequence. In a related embodiment of the present disclosure, the non-complementary portion of the nucleic acid template contains the desired alteration. In further embodiments the method comprises base pairing of the complementary regions of the nucleic acid sequence template with the nucleic acid sequence near the target DNA sequence, with the exception of the non-complementary portion. In yet further embodiments the method comprises incorporating the desired alteration into the target DNA sequence in a sequence-specific manner. In a related embodiment of the method, the MiniVector lacks both a bacterial origin of replication and an antibiotic selection gene. In some embodiments the MiniVector has a size up to about 2,500 base pairs.

In some embodiments, the present disclosure relates to a method of treating a genetic disorder, or other condition, in a subject in need thereof, where an alteration of a target DNA sequence is desired. In some embodiments, such a method comprises administering to a subject a therapeutically effective amount of a MiniVector comprising a nucleic acid sequence template. In related embodiments, the nucleic acid sequence template comprises at least one portion complementary to a nucleic acid sequence near the target DNA sequence; and at least one portion which is not complementary to the target DNA sequence, where the non-complementary portion of the nucleic acid template contains the desired alteration. In additional embodiments, the method comprises base pairing of the complementary regions of the nucleic acid sequence template with the nucleic acid sequence near the target DNA sequence, with the exception of the non-complementary portion. Further embodiments of the method comprise incorporating the desired alteration into the targeted DNA sequence in a sequence-specific manner. In a related embodiment of the method, the MiniVector lacks both a bacterial origin of replication and an antibiotic selection gene. In some embodiments the MiniVector has a size up to about 2,500 base pairs.

The above objects and other objects, features, and advantages of the present disclosure are readily apparent from the following detailed description of the best mode for carrying out the invention when taken in conjunction with the accompanying drawings.

### BRIEF DESCRIPTION OF THE DRAWINGS

In order that the manner in which the above recited and other advantages and objects of the invention are obtained, a more particular description of the invention briefly described above will be rendered by reference to specific embodiments thereof, which are illustrated in the appended Figures. Understanding that these Figures depict only typical embodiments of the invention and are therefore not to be considered limiting of its scope, the invention will be described with additional specificity and detail through the use of the accompanying Figures in which:

FIG. 1. shows preparation of MiniVector encoding template for DNA alteration;

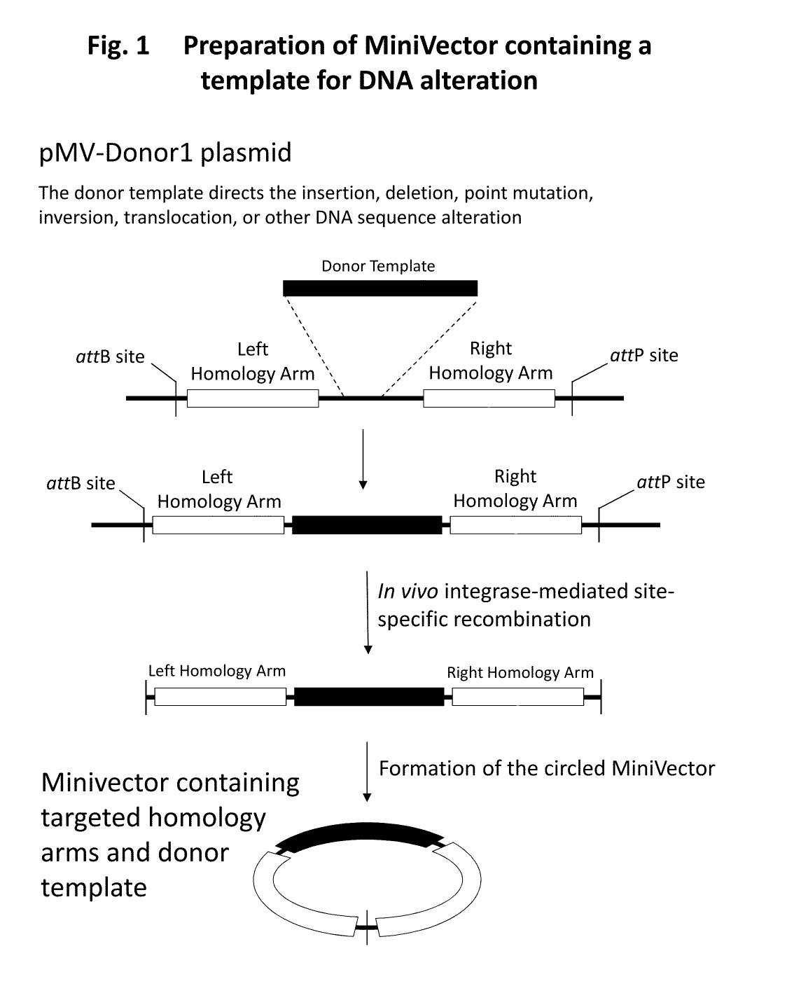

FIG. 2. shows targeted genome editing with MiniVector template;

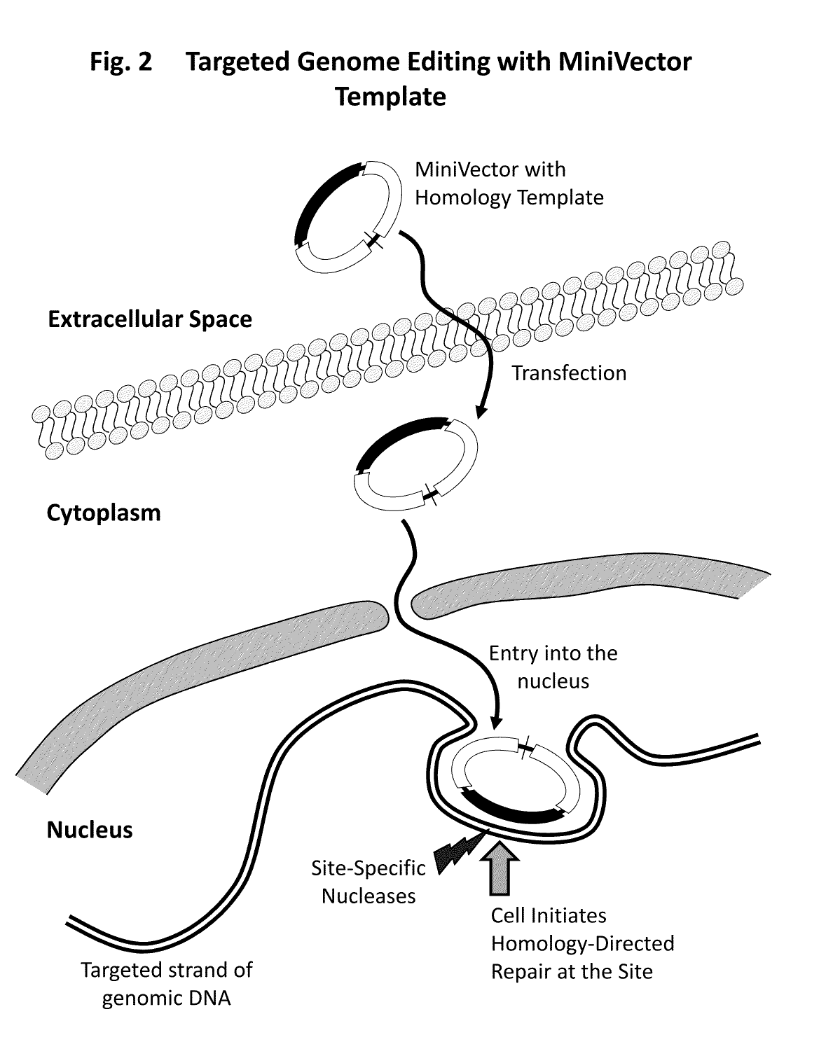

FIG. 3. shows an exemplary embodiment of zinc finger mediated gene editing with MiniVector as the repair template for modification of the IL2Rγ gene. Initial sequence of the wild-type, endogenous IL2Rγ gene is labeled to show location of the Kozak sequence and start codon. Non-complementary portion of the repair template shows added sequence for insertion of the restriction site into the IL2Rγ gene. As shown, this Xho site is encoded directly before the start codon for the gene. On the donor template, the sequence with the XhoI site that is to be inserted is flanked by two homology arms. These homology arms are complementary to the DNA sequence to the left and right of the site in the cellular genome that has been targeted for editing. A MiniVector is generated comprising the full length of the donor template;

FIGS. 4A-4B show the results of the PAGE (Polyacrylamide Gel Electrophoresis) analysis. The left three lanes are controls in which each of the three donor templates (either MiniVector at equi-mass, plasmid, or MiniVector at equi-moles) were delivered to the cell without any ZFNs (FIG. 4A). The next lanes show the experimental results when the ZFNs were delivered along with the plasmid-based donor template or the MiniVector-based donor template at either equi-mass or equi-molar amount compared to the amount of plasmid delivered (FIG. 4A). Determining the density of the two lower bands to the density of the top, uncut band provides a measure of the percentage of the total alleles that were successfully repaired. Analysis of the gel showed that MiniVectors were successful in targeting the alleles and inserting the restriction site (FIG. 4B); and

FIGS. 5A-5C show targeted gene correction by a MiniVector donor. MiniVector donor templates carried an intact 3′ region of the GFP gene, with an upstream functional P_{PGK} promoter (FIG. 5A) (left) or a truncated form of the same promoter (FIG. 5A) (right). The chromosomal target for repair was a transcribed GFP gene bearing an I-AniI site (yellow triangle) and two in-frame N-terminal stop codons (red lines) to prevent GFP expression (FIG. 5B) (above). This DNA target is integrated in the chromosome of HEK293T cells. Successful homology-directed repair corrects the GFP gene to generate GFP+ cells (FIG. 5B) (below) which contain a functional copy of the GFP gene in their chromosome. Flow cytometric analysis was performed on the HEK293 cells at 3 days post-transfection. Data is shown quantifying BFP+ cells (FIG. 5C) (top row) and GFP+ cells among the identified BFP+ cell population (FIG. 5C) (bottom row). Percentages of transfected BFP+ cells are shown above (BFP+ cells, expressing I-AniI). Percentage of the BFP+ cells also identified as GFP+ (successfully corrected by HDR) are shown (FIG. 5C) (below). Data from a control which was not transduced with I-AniI shows now BFP expression as expected (FIG. 5C) (left).

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The present disclosure provides methods and compositions for targeted DNA engineering to edit or alter DNA using the intrinsic cellular DNA repair machinery. The methods disclosed herein utilize a MiniVector as a template for homology-directed repair, alteration, or replacement of a target DNA sequence. The methods and compositions disclosed herein may be used to target any DNA sequence in any cell in vivo or in vitro, including but not limited to, any plant or animal cells, e.g., mammalian cells. The methods and compositions disclosed herein may be used with any cell type, including but not limited to, somatic cells and stem cells.

Targeted DNA engineering involves editing or altering the endogenous DNA within a cell in a directed manner at a specific site along the DNA within the cell. Genome editing or targeted DNA editing may be performed in any organism or cell including yeast, insects, invertebrates, mammals, fish, rodents, humans, plants, bacteria, and insects to name a few.^{1,2,8,12} Furthermore, targeted DNA editing may be performed in any cell type, including but not limited to stem cells and somatic cells. The endogenous DNA to be edited may be genomic DNA, mitochondrial DNA, or plastid DNA.¹³

Genome editing or targeted DNA engineering may be used for therapeutic purposes, such as to repair a genetic mutation, or may be used in basic research, for example to study the function of a specific genes.⁹⁻¹¹ Additionally, genome editing of plants, algae, bacteria, and archaea are being explored as new approaches for the development of food and biofuels.¹ Genetic modification through targeted DNA editing or altering provides an efficient and controlled method for producing plants with one or more desired characteristics, including characteristics that are normally not found in those crops, such as resistance to herbicides or pests, or nutritionally balanced food or feed products.

Gene therapy involves the delivery of DNA or RNA to a diseased organ or cells to correct, repair, replace, or alter defective genes or other DNA sequences implicated in disease. This may be achieved through a number of different approaches. If the disease state is a consequence of a missing or non-functional gene or other DNA sequence, a functional copy of the gene may be delivered to the disease locus. Gene expression may be controlled using RNA interference (RNAi) and RNA activation technologies such as small interfering RNA (siRNA), small activating RNA (saRNA), short hairpin RNA (shRNA), and microRNA (miRNA). In some embodiments, the present disclosure pertains to a method of using DNA MiniVectors for targeted DNA engineering for repairing, altering, replacing, adding, deleting, duplicating, or inverting a sequence of interest.

### DNA Repair Mechanisms

Cells have intrinsic mechanisms to attempt to repair any double or single stranded DNA damage. The cell repair mechanisms evolved to repair any DNA damage that is the result of natural causes. There are several ways to alter, repair, replace, add, delete, duplicate, and invert genomic, mitochondrial, chloroplastic, or extra-chromosomal DNA sequences. All these methods depend on DNA homology.

One way involves supplying the cell with a template that can be used in DNA homologous recombination. Recombination of this type depends upon a section of DNA with homology. The frequency of the event is increased by the induction of DNA damage (typically a double-strand break or nick) near the defective sequence and thus, the template will be used to recombine in, to thus fix or alter the defective sequence to the desired sequence encoded by the template. Double-strand breaks can be induced by a sequence specific endonuclease, such as meganuclease, zinc finger nuclease, or TAL nuclease. Nicks can be generated by a sequence specific nicking endonuclease.

Another way to repair or alter DNA sequences is to use an enzyme that will exchange a genomic, mitochondrial, chloroplastic, or extra-chromosomal sequence for the template. Rather than copying the template as in the first method, in this method, an enzyme, such as DRAP, will use the template to search the genome, mitochondria, chloroplast, or extra-chromosome(s) for the homologous region. When the homologous region is matched, DRAP will generate two sets of double-stranded breaks in both the template and target sequence, and will swap out the genomic, mitochondrial, chloroplastic, or extra-chromosomal sequence for the template in a “flip-in” mechanism.

Other DNA repair systems involve transposase, recombinase or integrases. Transposon systems such as the Sleeping Beauty transposase can also accomplish homologous recombination though a cut and paste mechanism. Integrase systems, such as HIV integrase, can add, delete, duplicate, or invert a sequence through homologous recombination. These cellular repair mechanisms may be exploited to direct the targeted and desired DNA alteration of the endogenous DNA sequence, including but not limited to: inversion, insertion, deletion, point mutation, duplication, or translocation.

### Delivery of Repair Template

Despite the tremendous therapeutic potential of gene therapy, and the large number of diseases identified as good candidates for gene therapy, the field has so far been largely unsuccessful. These failures are a consequence of complications associated with DNA delivery. Several approaches for introducing the repair template have been evaluated. The repair template may be introduced as a single stranded linear DNA, double stranded linear DNA, double stranded plasmid, or single stranded plasmid. Further, the repair template may be delivered as naked DNA or packaged within a viral delivery vehicle.^{1,4}

The most common DNA delivery methods utilize viral vectors. Delivery efficiency is high for viral vectors. However, viral vectors have limited therapeutic potential because of problems observed in clinical trials including toxicity, immune and inflammatory responses and difficulties in targeting and controlling dose. In addition there is justifiable concern that the vectors will integrate into the genome, with unknown long term effects, and the possibility that the virus may recover its ability to cause disease.

Much effort has therefore been directed towards non-viral vector systems, such as plasmid DNA. Linear DNA templates may be delivered using a plasmid. These vectors are attractive because they are simple to produce and store and they can stably persist in cells. However, there is a significant portion of the plasmid vector that is not a component of the homology arms or donor repair template. This is because plasmids are propagated in bacterial strains and thus are required to contain bacterial DNA sequences, notably a prokaryotic origin of replication and an antibiotic resistance marker for maintenance of the plasmid. The presence of these bacterial sequences has a number of very serious and deleterious consequences. Most notably, it limits how small the plasmids can be made. Large plasmids, of several thousand base pairs, are transfected at very low efficiency. Their large size also makes them susceptible to hydrodynamic shear forces associated with delivery (e.g., aerosolization) or in the bloodstream when introduced by intravenous delivery. Shear-induced degradation leads to a loss of biological activity that is at least partially responsible for the current lack of success in using non-viral vectors for gene therapy. Various cationic and liposomal transfection reagents have been designed to try and alleviate these problems with transfection, but these suffer from problems with cytotoxicity. Additionally, many human cells, including dendritic cells and T-cells, cannot be efficiently transfected with current plasmid vectors.

Reducing the size of DNA vectors appears to be a reasonable approach to increase cell transfection efficiency. One may envision that the bacterial sequences on the plasmid could be physically removed and resultant short linear DNA fragments that contain only the therapeutic sequences more easily introduced into cells than conventional plasmid vectors. However, linear DNA templates are unstable and rapidly degraded by the cells. Furthermore the ends of the linear DNA are highly reactive and trigger uncontrolled DNA repair and recombination process and additionally trigger an intrinsic immune response and cellular toxicity or apoptosis.

Hence, despite the tremendous potential of gene repair and homology-directed gene alteration, current approaches provide very low efficiency of repair or editing event and have the potential to introduce harmful or undesired DNA sequences and outcomes. Thus, there is a need for gene targeting therapies that are stable in biological environments and that allow for greater cell transfection and transgene expression.

### MiniVectors

The present disclosure relates to MiniVector for use as a template for homology-directed repair, alteration, or replacement. DNA MiniVectors (as small as ˜250 bp) display remarkable transfection efficiencies in all cell types tested, including cell types, such as suspension cells, T-cells, dendritic cells, that are typically recalcitrant to transfection with plasmids. There is great potential for use of this invention in gene replacement therapies and for genetic reprogramming of human diseased cells. Genomic, mitochondrial, chloroplastic, or extrachromosomal sequences that are mutated, needing repair, needing to be altered or replaced, needing to be added, deleted, duplicated, or inverted may be fixed in vivo using MiniVectors as a template for DNA corrections or as the piece of DNA that is inserted (“flipped in”) or integrated during the process known as gene replacement.

Supercoiled MiniVectors and methods for producing the MiniVectors have been described in U.S. application Ser. No. 11/448,590 (US Publication No.: US 20070020659 A1); Title: “Generation of Minicircle DNA with Physiological Supercoiling”, by Zechiedrich et al., which is incorporated herein by reference in its entirety. Additionally, As used herein “MiniVector” or supercoiled DNA vector system has also been previously described in U.S. application Ser. No. 12/905,612 (US Publication No.: US 20110160284 A1); Title: “Supercoiled MiniVectors for Gene Therapy Applications”, by Zechiedrich et al., which is also incorporated herein by reference in its entirety.

An embodiment of the present disclosure provides for a small, supercoiled DNA MiniVectors that are non-viral gene-therapy vectors, which are almost completely devoid of bacterial sequences for use as a template for homology-directed repair, alteration, or replacement. Because of their small size, these MiniVectors are transfected with high efficiency. The lack of bacterial sequence allows for an optimal donor template design containing only the desired DNA sequence in a double stranded and supercoiled, bioactive form.

In an embodiment, the present disclosure relates to a MiniVector comprising a nucleic acid sequence template for homology-directed repair, alteration, or replacement of the targeted DNA sequence within a cell in vivo or in vitro. In some embodiments of the present disclosure, the nucleic acid sequence template of the composition described above may comprise at least one portion of the template complementary to a nucleic acid sequence near the targeted DNA sequence; and at least one portion of the template which is not complementary to the targeted DNA sequence. In a related embodiment, the non-complementary portion of the nucleic acid template may contain the alteration desired in the targeted DNA sequence. The findings described herein demonstrate that the MiniVectors possess advantages for targeted genomic editing or repair.

A MiniVector may be obtained in E. coli by in vivo integrase-mediated site-specific recombination. It contains, for example, a nucleic acid molecule with merely the transgene expression cassette (including promoter and a nucleic acid sequence, wherein the nucleic acid sequence may be, for example, a template for homology-directed repair, alteration, or replacement of the targeted DNA sequence, and, importantly, no bacterial-originated sequences. (Mali et al., 2013; Alexander B L et al., 2007; Alton et al., 2007)

Minivectors used for targeted DNA alteration may be double-stranded, circular DNA molecules of the size of from about 100 base pairs (bp) to about 2.5 kilo base (kb), such as from about 200 bp to about 2.2 kb, for example from about 300 bp to about 2.0 kb, for example from about 400 bp to about 1.9 kb, for example from about 500 bp to about 1.8 kb, for example from about 600 bp to about 1.7 kb, for example from about 700 bp to about 1.6 kb, for example from about 800 bp to about 1.5 kb, for example from about 900 bp to about 1.4 kb, for example from about 1 kb to about 1.3 kb, for example from about 1.1 kb to about 1.2 kb. MiniVectors can be made in size increments of about 100 bp or fewer.

In an embodiment of the present disclosure, the MiniVector, of the composition described above, may lack both a bacterial origin of replication and an antibiotic selection gene. In a related embodiment the MiniVector may be of a size up to about 2,500 base pairs. In some embodiments, the MiniVector may further comprise a chemical moiety, a modified oligonucleotide, and/or a modified backbone.

The MiniVectors may be labeled, e.g., using a chemical moiety, as desired. Representative labels include fluorescein, biotin, cholesterol, dyes, modified bases and modified backbones. Representative dyes include: 6-carboxyfluorescein, 5-/6-carboxyrhodamine, 5-/6-Carboxytetramethylrhodamine, 6-Carboxy-2′-,4-,4′-,5′-,7-,7′-hexachlorofluorescein), 6-Carboxy-2′-,4-,7-,7′-tetrachlorofluorescein), 6-Carboxy-4′-,5′-dichloro-2′-,7′-dimethoxyfluorescein, 7-amino-4-methylcoumarin-3-acetic acid), Cascade Blue, Marina Blue, Pacific Blue, Cy3, Cy5, Cy3.5, Cy5.5, IRDye700, IRDye800, BODIPY dye, Texas Red, Oregon Green, Rhodamine Red, Rhodamine Green. Additional modifications may also include modified bases (e.g. 2-aminopurine, methylated bases), or modified backbones (e.g., phosphorothioates, where one of the non-bridging oxygen is substituted by a sulfur; 2′-O-methyl-RNA-oligonucleotides; methyl-phosphate oligonucleotides). Multiple labels, including chemical moieties and/or modified bases and/or modified backbones, may be used simultaneously, if desired. Methods of labeling nucleotides are described, for example, in “Nucleic Acid Probe Technology” by Robert E. Farrell; RNA Methodologies (Third Edition), 2005, pp. 285-316; and “Enzymatic Labeling of Nucleic Acids” by Stanley Tabor and Ann Boyle, in Current Protocols in Immunology 2001 May; Chapter 10: Unit 10.10. The teachings of these references are incorporated herein by reference in their entirety.

### Nucleases

As mentioned above, targeted DNA engineering may be desired for a number of different intended outcomes, for example to repair a mutation, introduce a mutation, introduce a new gene, reprogram the cell, delete a portion of the DNA sequence, alter gene expression patterns, etc. (Perez-Pinera et al., 2012). This process may typically involve the use of engineered site-specific nucleases. In an exemplary embodiment of the present disclosure, the site-specific nuclease may be encoded by a portion of the nucleic acid sequence template of the MiniVector described above. In another embodiment, the site-specific nuclease may be encoded by a separate MiniVector, a plasmid, a messenger RNA, or a virus, or may be delivered as a protein.

These engineered nucleases may contain two, fused domains each with a separate function. The first domain, a DNA binding domain, may be engineered to recognize and bind to a specific DNA sequence. The second domain may consist of a nuclease, or enzyme capable of making a double or single stranded break in the DNA. When introduced into a cell, the engineered nuclease may bind to the cellular DNA if the targeted sequence is present. Upon binding, the nuclease may cause a cleavage or break in the backbone of the DNA. Hence, the cleavage may be designed to affect both strands of the double helix (double stranded break, DSB) or it can be designed to affect only one stand (single stranded break, SSB) based on the engineered activity of the nuclease domain. The nuclease by itself will cleave DNA nonspecifically, however when fused to a DNA binding domain the nuclease activity will be directed toward a specific site. These engineered nucleases, therefore are also referred to as site-specific nucleases. (Jensen et al., 2011; Joung and Sander, 2013; Perez-Pinera et al., 2012). It should be noted that homologous repair via recombination may also occur in the absence of any engineered nuclease, or could be performed with a nonspecific nuclease. (Jensen et al., 2011). The rate of homologous recombination mediated gene editing without a site-specific nuclease, however, is exceptionally low. (Mali & Cheng, 2012; Parekh-Olmedo et al., 2005; Perez-Pinera et al., 2012).

Multiple types of engineered nucleases have been developed. For instance, the first and most studied was based on a zinc finger binding domain fused to a nuclease, and is referred to as a zinc-finger nuclease (ZFN). (Carrol, 2008; Jensen et al., 2011; Joung & Sander, 2013; Perez-Pinera., 2012). Another approach utilizes a nuclease fused to a DNA binding domain derived from transcription activator-like effectors (TALEs) to give rise to a TALEN, or transcription activator-like effector nuclease. (Joung & Sander, 2013; Perez-Pinera., 2012). An additional approach utilizes a bacterial system that has been repurposed for genome editing in mammalian and other non-bacterial organisms. In this system, Clustered Regularly Interspaced Short Palindromic Repeats (CRIPSR) RNAs work in a complex with CRISPR-associated (Cas) proteins to direct cleavage of a DNA sequence matching the CRISPR RNA sequence. In one example, the Cas9 protein was fused to a CRISPR RNA targeting the desired DNA sequence to be cleaved. (Mali et al., 2013). Any one or combination of these site-specific nuclease systems may be combined with a donor, or DNA template to direct the subsequent repair as desired.

When an artificial break is caused by an engineered nuclease, these cellular repair mechanisms may be exploited to direct the repair process in order to cause any number of alterations to the endogenous DNA sequence, including but not limited to: inversion, insertion, deletion, point mutation, duplication, or translocation. (Joung and Sander, 2013; Perez-Pinera et al., 2012; Parekh-Olmedo, Ferrara, Brachman, & Kmiex, 2005) In order to direct the repair event toward a desired outcome, a template DNA strand may also be introduced into the cell along with the engineered nucleases. This template strand may be single or double stranded, and will have portions that are complementarily, or homologous to the cellular DNA at or near the site of the induced DNA cleavage event that is mediated by the site-specific nuclease. In one example, the template strand may have two regions of homology that are complementary to the DNA sequence on either side of a double stranded break. In between these homology arms, a template region may be present that corresponds to the desired final sequence of the cellular DNA following repair. The template region may in one example, encode a nucleic acid sequence to be inserted into the endogenous DNA. It could alternatively encode a single point mutation or any number of other alterations to the nucleic acid sequence. (Joung and Sander, 2013; Perez-Pinera et al., 2012; Jensen et al., 2011).

In addition to engineered nucleases, a DNA binding domain may alternatively be fused to an integrase or recombinase domain in order to direct site-specific recombination with a repair template. (Perez-Pinera et al., 2012). In another embodiment, transposase or recombinase-mediated gene alteration could be performed with the repair template by using a system such as sleeping beauty transposon. (Richardson et al., 2002).

In an embodiment, this disclosure provides a composition for targeted alteration of a DNA sequence comprising a MiniVector comprising a nucleic acid sequence template for homology-directed repair, alteration, or replacement of the targeted DNA sequence within a cell in vivo or in vitro. In an exemplary embodiment the targeted DNA sequence to be altered may be genomic, mitochondrial, or plastid DNA within the cell. In some embodiments, the cell may be a mammalian, prokaryotic, eukaryotic, archaea, or plant cell. In another embodiment, the cell may be a somatic cell, germ cell, or a stem cell.

In some embodiments of the present disclosure, the nucleic acid sequence template of the composition described above may comprise at least one portion of the template complementary to a nucleic acid sequence near the targeted DNA sequence; and at least one portion of the template which is not complementary to the targeted DNA sequence. In a related embodiment, the non-complementary portion of the nucleic acid template may contain the alteration desired in the targeted DNA sequence.

In some embodiments the present disclosure pertains to a kit comprising a MiniVector comprising a nucleic acid sequence template for homology-directed repair, alteration, or replacement of the targeted DNA sequence within a cell in vivo or in vitro.

In a further embodiment, the composition described above further comprises at least one site-specific nuclease. In some embodiments the present disclosure pertains to a kit comprising the aforementioned composition. In an exemplary embodiment of the present disclosure, the site-specific nuclease may be encoded by a portion of the nucleic acid sequence template of the MiniVector. In another embodiment, the site-specific nuclease may be encoded by a separate MiniVector, a plasmid, a messenger RNA, or a virus, or may be delivered as a protein. The site-specific nuclease may be selected from a group consisting of zinc finger nuclease (ZFN), transcription-activator-like effector nuclease (TALEN), meganuclease, and CRISPR (clustered regularly interspaced short palindromic repeats)/CAS (CRISPR associated) system.

In an embodiment, the site-specific nuclease may induce one or more single stranded breaks in the target DNA sequence. In another embodiment, the site-specific nuclease may induce one or more double stranded breaks in the target DNA sequence. In an embodiment of the present disclosure, the homology-directed repair, alteration, or replacement may be mediated by a transposase or recombinase, including but not limited to sleeping beauty transposon system.

Another embodiment of the present disclosure relates to a method of altering a target DNA sequence in a cell using the composition(s) described above. Such a method may comprise transfecting a MiniVector comprising a nucleic acid sequence template. In some embodiments, the nucleic acid sequence template may comprise at least one portion complementary to a nucleic acid sequence near the target DNA sequence; and at least one portion which is not complementary to the target DNA sequence. In related embodiments, the non-complementary portion of the nucleic acid template contains the desired alteration. Such a method further comprises base pairing of the complementary regions of the nucleic acid sequence template with the nucleic acid sequence near the target DNA sequence, with the exception of the non-complementary portion; and incorporating the desired alteration into the target DNA sequence in a sequence-specific manner.

In an additional embodiment, such a method may further comprise the step of providing at least one site-specific nuclease. The site-specific nuclease may be encoded by a portion of the nucleic acid template of the MiniVector. In an exemplary embodiment, the step of providing the site-specific nuclease may comprise co-transfecting a separate MiniVector, a plasmid, a messenger RNA, or a virus encoding the site-specific nuclease, or a protein. The site-specific nuclease may be selected from a group consisting of zinc finger nuclease (ZFN), transcription-activator-like effector nuclease (TALEN), meganuclease, and CRISPR (clustered regularly interspaced short palindromic repeats)/CAS (CRISPR associated) system. In an embodiment, the site-specific nuclease may induce one or more single stranded breaks in the target DNA sequence. In another embodiment, the site-specific nuclease may induce one or more double stranded breaks in the target DNA sequence. In some embodiments, the alteration of the target DNA is mediated by a transposase or recombinase, including but not limited to sleeping beauty transposon system.

In yet another embodiment, the present disclosure pertains to a method of treating a genetic disorder, or other condition, in a subject in need thereof, where an alteration of a target DNA sequence is desired. In an embodiment, the subject may be a mammal or a plant. In some embodiments, such a method comprises administering to a subject a therapeutically effective amount of the composition(s) described above. In another embodiment, the method may further comprise co-administering at least one site-specific nuclease. In some embodiments, the site-specific nuclease is encoded by a portion of the nucleic acid template of the MiniVector. In another embodiment, the co-administering may comprise of providing a separate MiniVector, plasmid, a messenger RNA, or a virus encoding the site-specific nuclease or providing a protein.

The site-specific nuclease may be selected from a group consisting of zinc finger nuclease (ZFN), transcription-activator-like effector nuclease (TALEN), meganuclease, and CRISPR (clustered regularly interspaced short palindromic repeats)/CAS (CRISPR associated) system. In an embodiment, the site-specific nuclease may induce one or more single stranded breaks in the target DNA sequence. In another embodiment, the site-specific nuclease may induce one or more double stranded breaks in the target DNA sequence. In some embodiments, the alteration of the target DNA is mediated by a transposase or recombinase, including but not limited to sleeping beauty transposon system.

Possible variations and modifications to the methods and compositions disclosed herein may be envisioned. For example, to control cellular location (e.g., mitochondrial, chloroplastic, or extra-chromosomal versus nuclear), special DNA sequences that “tag” or direct the MiniVectors to the desired cellular location may be included. To target specific cells in a mixed population of cells (e.g., lung fibroblasts in living mice), various moieties (epitopes, fatty acids, special protein sequences, etc.) may be affixed to MiniVectors to target them to cells that have, for example, receptors that bind the moiety or are more susceptible to DNA uptake when the DNA is attached to the moiety, etc. MiniVectors may also be non-covalently complexed with delivery vehicles such as transfection agents or targeting moieties such as polymers or proteins.

### Applications and Advantages

Current plasmid vectors are fairly large (>5 kb) and thus not suitable for the new applications because of (i) Size: the vector becomes even larger with the sequence of interest,; (ii) Transfection efficiency: plasmids have poor transfection that little to no template DNA is delivered inside the cell for repair; and (iii) Nonspecific DNA: bacterial sequences are problematic and can cause silencing of the transgene. DNA MiniVectors are much smaller and offer the advantages of higher transfection, increased stability, and improved delivery of DNA repair templates that are less likely to be silenced.

In addition to therapeutic applications, gene or DNA replacement, repair, and alteration may also be applied in non-therapeutic applications. For example, it may be used to generate transgenic organisms such as knock-out mice, or it may be used to alter cells such as for immortalization of a cell line. These transgenic cells and organisms may have utility as disease models and in the study of the DNA function. Furthermore, transgenic organisms may have commercial usefulness for example in agriculture where alteration, repair, insertion, deletion, duplication, or inversion of a gene may provide novel beneficial characteristics such as disease or pest resistance. For all of these applications, MiniVectors provide an improved platform over traditional plasmids or viral vectors due to their improved efficiency of transfection and ease of synthesis. It can further be expected that MiniVectors will provide a better repair template since all non-relevant sequences (bacterial origin and antibiotic resistance) will have been removed and are therefore not able to interfere with homologous binding to host DNA.

### Additional Embodiments

Reference will now be made to various embodiments of the present disclosure and experimental results that provide support for such embodiments. Applicants note that the disclosure herein is for illustrative purposes only and is not intended to limit the scope of the claimed subject matter in any way.

### EXAMPLE 1

Nucleofection and Targeted Gene Editing with MiniVectors

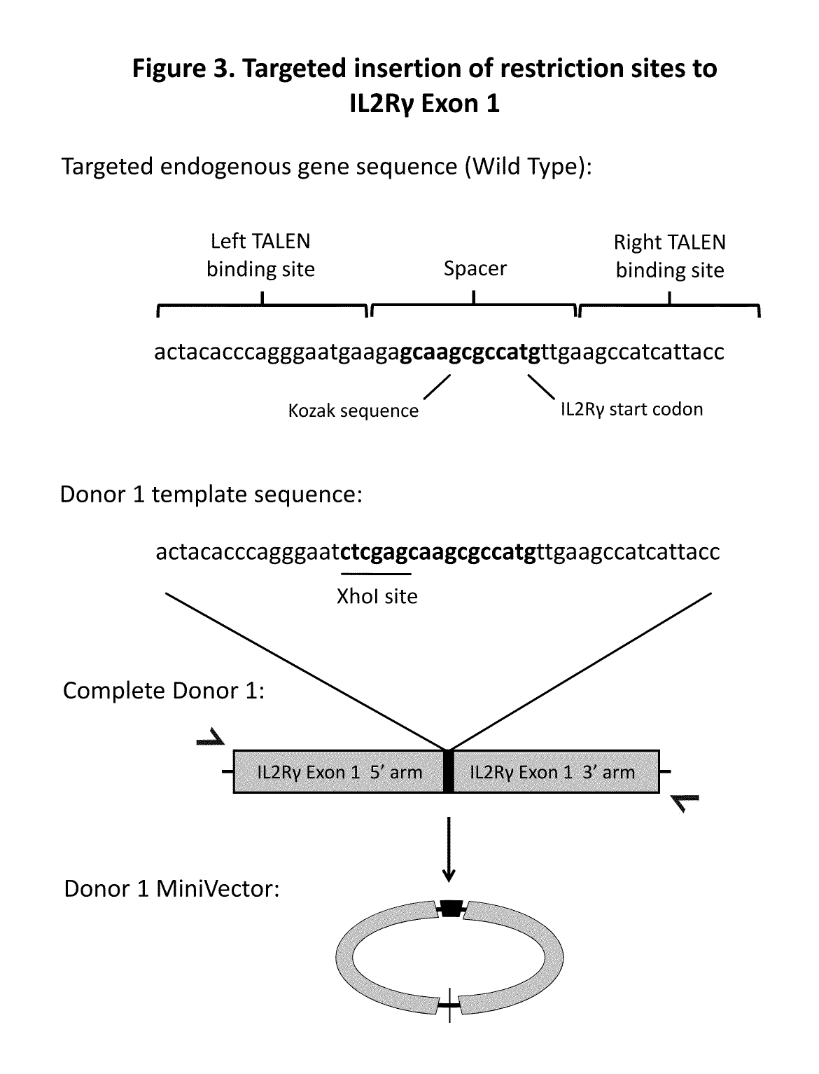

To demonstrate that MiniVectors can serve as a donor template for ZFN-mediated targeted gene editing, an experiment was conducted in which the modification was designed to produce an easily detected and quantifiable change in the genomic DNA of the cell. The donor template is designed with two homology regions complementary to the first portion of the IL2Rγ gene. In between the two homology regions is a template sequence containing a site that can be recognized by the XhoI restriction enzyme. The initial, proof of concept experiment was conducted with K562 cells (a non-adherent, leukemia cell line) using the Lonza, Nucleofector™ transfection system to deliver the DNA. It should be noted, however, that any approach (transfection agent, electroporation, etc.) could be used for DNA delivery to the cell. Following the nucleofection of DNA and ZFNs, the genomic DNA of the cells was harvested and analyzed. To detect the insertion of the XhoI site, appropriate primers were used with PCR to amplify the segment of genomic DNA containing the DNA sequence targeted for editing. This amplified PCR product was then subjected to a restriction digest with the XhoI enzyme (FIG. 3). When run on a gel, the PCR product will either remain as a single larger band (uncut, therefore un-edited) or will run as two smaller bands (cut, therefore successfully edited with the donor template). Ratio of the large bands to the two smaller bands permitted a quantitative assessment of successful gene targeting. In the experiment, the donor template was delivered either on a traditional plasmid, or on a MiniVector. The much smaller MiniVector will provide many more template molecules if used in an equivalent mass amount to the larger plasmid. Therefore two doses of MiniVectors were compared to the plasmid template: equivalent mass amount of MiniVectors, and a lower dose which was calculated to deliver an equivalent molar quantity of the MiniVectors.

### Methods of Cell Culture

K562 cells (human immortalized myelogeneous leukemia cell line, ATCC) were maintained in RPMI media incubated at 37° C. and 5% CO₂. Media was exchanged every 2-3 days and subculturing was performed when the cell concentration reached 1×10⁶ cells/mL. To prepare for the experiment, cells were split to 200,000 cells/mL and maintained between 200,000 to 500,000 cells/ml for 3 days such that cells were in log phase growth for the duration of the time period.

### Methods for Nucleofection of MiniVectors and Plasmid DNA

In 6 well plates, 3 ml RPMI media per well was pre-warmed to 37° C. For each sample, 1×10⁶ cells were spun down at 800 RPM and 5° C. Nucleofection solution was prepared by combining 1 ml of Nucleofection Solution II (Lonza) with 1 aliquot of Nucleofection Solution I (Lonza). Cell pellets were resuspended in 100 μl nucleofection solution per 1×10⁶ cells. To the resuspended cells, a sample of either MiniVector or plasmid DNA was added and mixed well. The final mixture of cells and DNA (about 100 to 110 μl volume) was added to a blue-top transfection cuvette (Lonza). Nucleofection was performed with a 2D Nucleofector™ (Lonza) using program 2-16. Note that cuvettes were tapped on the bench top prior to starting so that the sample was fully resting in the bottom of the cuvette. Immediately after nucleofection was complete, 500 μl warm media was added to the cuvette. Finally samples were transferred from the cuvette to the pre-warmed 6 well plate. Cuvettes were also rinsed with 500 μl of media and added to the well. Plates were cultured from 3 to 14 days at 37° C. and 5% CO₂ in an incubator.

On Day 3 post-nucleofection, cells were analyzed by FACS to measure transfection efficiency. On day 14 post-nucleofection, gDNA was collected from the cells using published methods. (Urnov et al., 2005; Zou et al., 2009). The genomic locus of interest was targeted and amplified by PCR with appropriately designed primers. The PCR product was then digested with restriction enzyme XhoI (NEB) which will cut at the inserted restriction site if genome editing was successful. The digested PCR products for each sample are finally analyzed by PAGE to determine the percentage of targeted alleles. Percentage of targeted alleles was calculated by comparing the density of the cut band vs. the uncut band.

### Results of Targeted Gene Editing with MiniVectors and Plasmids

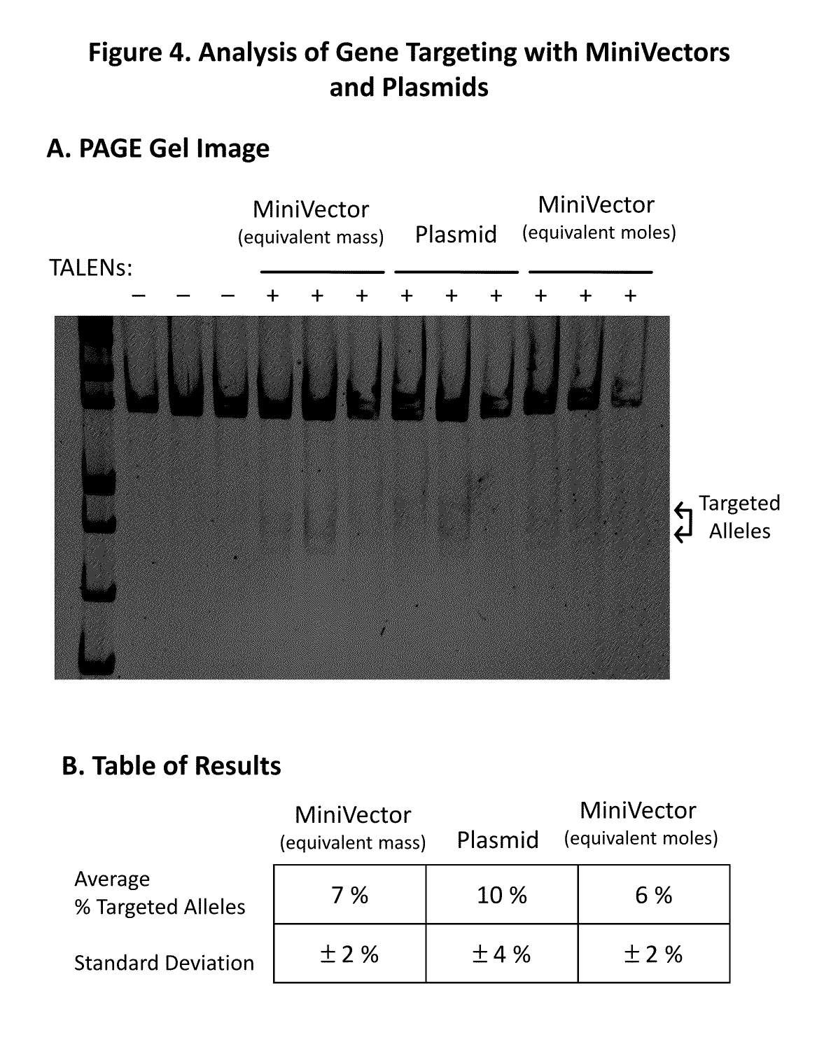

PAGE was used to determine the percentage of targeted alleles within the cell population. If targeted genome editing was successful, a restriction site for the XhoI restriction enzyme was created in within the targeted DNA of the IL2Rγ gene. This targeted region of DNA was amplified by PCR with appropriate primers and then subjected to restriction digest with XhoI enzyme. When run on the gel, the PCR product of those alleles that were not modified ran as a single, larger band since they did not contain the restriction site and were not cut by the enzyme. In contrast, any alleles that were targeted and repaired with the donor template ran as two shorter bands since the PCR product was recognized and cut by the enzyme. The MiniVector was successful in providing the donor template and enabled 6% to 7% of alleles to be modified with the restriction site (FIG. 4).

### EXAMPLE 2

### Targeted Gene Correction Using MiniVector-Based Donor Template and Meganuclease

A standard assay was used to demonstrate the ability of MiniVectors to serve as a donor template in combination with a homing endonuclease (a rare-cutting meganuclease) for targeted gene correction (TGC). (Humbert and Maizels, 2012). Gene correction was achieved in a cell culture system with HEK293T cells (human embryonic kidney cells) that had been engineered to carry a stably integrated, but mutant form of the enhanced Green Fluorescent Protein (eGFP) gene under the control of a PGK (phosphoglycerate kinase) promoter. This mutant form of the eGFP gene bears two in-frame N-terminal stop codons to prevent expression of the fluorescent reporter protein in the cell line (GFP). MiniVectors were generated which provided a template for the fully functional eGFP expression cassette (both the PGK promoter and eGFP gene lacking the stop codons). This MiniVector therefore contained regions of DNA complementary to the expression cassette in the cellular genome, and a smaller region corresponding to the targeted DNA sequence in the cell that was not complementary since it did not contain the two stop codons. Two forms of the donor template MiniVector were tested here, one with the full length of the PGK promoter (a longer left homology arm) and one with a truncated PGK promoter sequence (shorter left homology arm). Both forms were transfected separately into cultures of HEK293 cells. Simultaneously, the cells were transduced with an integration-deficient lentivirus vector to deliver a rare-cutting nuclease (I-AniI, of the meganuclease family) which generates a double-strand break at a 20 bp target site near the location of the two stop codons in the eGFP gene. When successful homology-directed repair (HDR) occurs, the eGFP gene is corrected and the cells begin to express the GFP reporter (GFP+).

A separate fluorescent protein reporter system was used to identify the cells which had been successfully transduced by the virus and were producing the I-AniI nuclease. For this system the I-AniI nuclease was joined via a T2A peptide translational linker to mTagBFP (monomeric blue fluorescent protein). This allowed both the I-AniI protein and the mTagBFP reporter protein to be expressed concurrently from the same open reading frame. The two proteins were post-translationally separated by a cleavage event at the T2A peptide, allowing proper protein folding and functionality. Cells expressing the blue fluorescent protein (BFP+) could therefore be confirmed as also expressing the I-AniI nuclease. For all experiments, the cell populations were analyzed by flow cytometry. Protocols were followed as previously described. (Humbert and Maizels, 2012).

### Method

HEK293 cells were cultured as an adherent population in Eagle's Minimum Essential Medium (EMEM) supplemented with fetal bovine serum (FBS) at 10% and incubated at 37° C. and 5% CO₂. Media was replenished every 2-3 days and cells were passaged when confluency neared 80%. Cells were transfected with MiniVector donor templates and simultaneously transduced with the integration-deficient lentiviral vector encoding the I-AniI meganuclease and mTagBFP reporter. At 3 days post-transfection, cells were collected and analyzed by flow cytometry to determine which population was positive for the fluorescent reporter proteins.

### Results

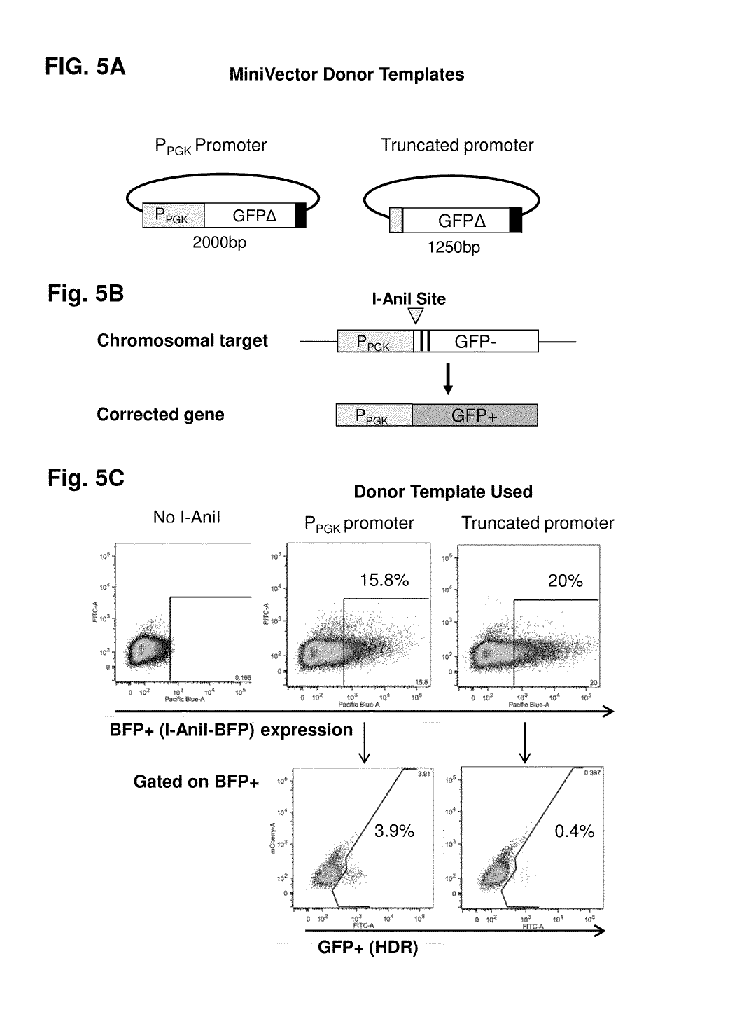

It was shown that there was no repair in the absence of I-AniI expression (FIG. 5C, top left), as expected. In cells expressing I-AniI (BFP+), the MiniVector bearing the intact PGK promoter supported targeted gene correction in 3.9% of transfected cells. This is comparable to the frequency of repair with a traditional plasmid donor. The frequency of targeted gene correction was 10-fold lower (0.4%) using a MiniVector donor with the truncated form of the PGK promoter; traditional vectors show a similar dependence on the full length promoter (FIG. 5C).

The embodiments described herein are to be construed as illustrative and not as constraining the remainder of the disclosure in any way whatsoever. While the embodiments have been shown and described, many variations and modifications thereof can be made by one skilled in the art without departing from the spirit and the teachings of the invention. Accordingly, the scope of protection is not limited by the description set out above, but is only limited by the claims, including all equivalents of the subject matter of the claims. The disclosure of all patents, patent applications and publications cited herein are hereby incorporated herein by reference, to the extent that they provide procedural or other details consistent with and supplementary to those set forth herein.

### REFERENCES

1. Alexander, B. L., Ali, R. R., Alton, E. W. F., Bainbridge, J. W., Braun, S., Cheng, S. H., Flotte, T. R., et al. (2007). Progress and prospects: gene therapy clinical trials (part 1). Gene therapy, 14(20), 1439-47. doi:10.1038/sj.gt.3303001
2. Alton, E., Ferrari, S., Griesenbach, U., Aiuti, A., Bachoud-Lévi, a C., Blesch, A., Brenner, M. K., et al. (2007). Progress and prospects: gene therapy clinical trials (part 2). Gene therapy, 14(22), 1555-63. doi:10.1038/sj.gt.3303033
3. Carroll, D. (2008). Progress and prospects: zinc-finger nucleases as gene therapy agents. Gene therapy, 15(22), 1463-8. doi:10.1038/gt.2008.145
4. Jensen, N. M., Dalsgaard, T., Jakobsen, M., Nielsen, R. R., Sørensen, C. B., Bolund, L., & Jensen, T. G. (2011). An update on targeted gene repair in mammalian cells: methods and mechanisms. Journal of biomedical science, 18(1), 10. doi:10.1186/1423-0127-18-10
5. Joung, J. K., & Sander, J. D. (2013). TALENs: a widely applicable technology for targeted genome editing. Nature reviews. Molecular cell biology, 14(1), 49-55. doi:10.1038/nrm3486
6. Kyriakouli, D. S., Boesch, P., Taylor, R. W., & Lightowlers, R. N. (2008). Progress and prospects: gene therapy for mitochondrial DNA disease. Gene therapy, 15(14), 1017-23. doi:10.1038/gt.2008.91
7. Mali, P., & Cheng, L. (2012). Concise review: Human cell engineering: cellular reprogramming and genome editing. Stem cells (Dayton, Ohio), 30(1), 75-81. doi:10.1002/stem.735
8. Mali, P., Yang, L., Esvelt, K. M., Aach, J., Guell, M., DiCarlo, J. E., Norville, J. E., et al. (2013). RNA-guided human genome engineering via Cas9. Science (New York, N.Y.), 339(6121), 823-6. doi:10.1126/science.1232033
9. Nakayama, M. (2010). Homologous recombination in human iPS and ES cells for use in gene correction therapy. Drug discovery today, 15(5-6), 198-202. doi:10.1016/j.drudis.2010.01.006
10. Parekh-Olmedo, H., Ferrara, L., Brachman, E., & Kmiec, E. B. (2005). Gene therapy progress and prospects: targeted gene repair. Gene therapy, 12(8), 639-46. doi:10.1038/sj.gt.3302511
12. Perez-Pinera, P., Ousterout, D. G., & Gersbach, C. a. (2012). Advances in targeted genome editing. Current opinion in chemical biology, 16(3-4), 268-77. doi:10.1016/j.cbpa.2012.06.007
13. Porteus, M. (2011). Homologous recombination-based gene therapy for the primary immunodeficiencies. Annals of the New York Academy of Sciences, 1246, 131-40. doi:10.1111/j.1749-6632.2011.06314.x
14. Richardson, P. D., Augustine, L. B., Kren, B. T. and Steer, C. B., Gene Repair and Transposon-mediated gene repair. Stem Cells (Dayton, Ohio) 30, 75-81 (2002).
15. Urnov, F. D., Miller, J. C., Lee, Y.-L., Beausejour, C. M., Rock, J. M., Augustus, S., Jamieson, A. C., et al. (2005). Highly efficient endogenous human gene correction using designed zinc-finger nucleases. Nature, 435(7042), 646-51. doi:10.1038/nature03556
16. Zou, J., Maeder, M. L., Mali, P., Pruett-Miller, S. M., Thibodeau-Beganny, S., Chou, B.-K., Chen, G., et al. (2009). Gene targeting of a disease-related gene in human induced pluripotent stem and embryonic stem cells. Cell stem cell, 5(1), 97-110. doi:10.1016/j.stem.2009.05.023
17. Smith, A. M. Takeuchi, R., Pellenz, S., Davis, L., Maizels, N., Monnat, Jr., R. J., and Stoddard, B. L. (2009). Generation of a nicking enzyme that stimulates site-specific gene conversion from the IAniI LAGLIDADG homing endonuclease. Proc. Nat. Acad. Sci. USA. 106: 5099-5104.
18. Davis, L., and Maizels, N. (2011). DNA nicks promote efficient and safe targeted gene correction. PLoS ONE, 6: e23981.
19. Fogg, J. M., Kolmakova, N., Rees, I., Magonov, S., Hansma, H., Perona, J. J., and Zechiedrich, E. L. (2006). Exploring writhe in supercoiled minicircle DNA. J Phys: Condens Matter 18: S145-S159.
20. Zhao, N., Fogg, J. M., Zechiedrich, L., and Zu, Y. (2011). Transfection of shRNA-encoding Minivector DNA of a few hundred base pairs to regulate gene expression in lymphoma cells. Gene Ther 18: 220-224.
21. Catanese, D. J., Jr., Fogg, J. M., Schrock, D. E., I I, Gilbert, B. E., and Zechiedrich, L. (2012). Supercoiled Minivectors DNA resist shear forces associated with gene therapy delivery. Gene Ther. 19: 94-100.
22. Humbert, O. and Maizels, N. 2012. Epigenetic modification of the repair donor regulates targeted gene correction. Mol. Therapy Nucleic Acids 1:e49.

What is claimed is:

1. A composition for targeted alteration of a DNA sequence comprising:
a MiniVector comprising a nucleic acid sequence template for homology-directed repair, alteration, or replacement of the targeted DNA sequence within a cell in vivo or in vitro, wherein the MiniVector lacks both a bacterial origin of replication and an antibiotic selection gene, and wherein the MiniVector has a size up to about 2,500 base pairs.

2. The composition of claim 1, wherein the nucleic acid sequence template for the homology-directed repair, alteration, or replacement of the targeted DNA sequence comprises:
at least one portion of the template complementary to a nucleic acid sequence near the targeted DNA sequence to be altered; and
at least one portion of the template which is not complementary to the targeted DNA sequence to be altered, wherein the non-complementary portion of the nucleic acid template contains the alteration desired in the targeted DNA sequence.

3. The composition of claim 1, wherein the targeted DNA sequence to be altered is genomic, mitochondrial, or plastid DNA within the cell.

4. The composition of claim 1, further comprising at least one site-specific nuclease.

5. The composition of claim 4, wherein the site-specific nuclease is encoded by a portion of the nucleic acid sequence template of the MiniVector.

6. The composition of claim 4, wherein the site-specific nuclease is encoded by a separate MiniVector, a plasmid, a messenger RNA, or a virus, or is delivered as a protein.

7. The composition of claim 4, wherein the site-specific nuclease is selected from a group consisting of zinc finger nuclease (ZFN), transcription-activator-like effector nuclease (TALEN), meganuclease, and CRISPR (clustered regularly interspaced short palindromic repeats)/CAS (CRISPR associated) system.

8. The composition of claim 4, wherein the site-specific nuclease induces one or more single stranded breaks in the target DNA sequence.

9. The composition of claim 4, wherein the site-specific nuclease induces one or more double stranded breaks in the target DNA sequence.

10. The composition of claim 1, wherein the homology-directed repair, alteration, or replacement is mediated by a transposase or recombinase, including but not limited to the sleeping beauty transposon system.

11. The composition of claim 1, wherein the MiniVector further comprises a chemical moiety, a modified oligonucleotide, and/or a modified backbone.

12. The composition of claim 1, wherein the cell is a mammalian, prokaryotic, eukaryotic, archaea, or plant cell.

13. A cell comprising the composition of claim 1.

14. The cell of claim 13, wherein the cell is a mammalian, prokaryotic, eukaryotic, archaea, or plant cell.

15. A kit comprising the composition of claim 1.

16. A kit comprising the composition of claim 4.

17. A method of altering a target DNA sequence in a cell comprising:
a) transfecting a MiniVector comprising a nucleic acid sequence template, wherein the nucleic acid sequence template comprises at least one portion complementary to a nucleic acid sequence near the target DNA sequence; and at least one portion which is not complementary to the target DNA sequence, wherein the non-complementary portion of the nucleic acid template contains the desired alteration;
b) base pairing of the complementary regions of the nucleic acid sequence template with the nucleic acid sequence near the target DNA sequence, with the exception of the non-complementary portion; and
c) incorporating the desired alteration into the target DNA sequence in a sequence-specific manner,
wherein the MiniVector lacks both a bacterial origin of replication and an antibiotic selection gene, and wherein the MiniVector has a size up to about 2,500 base pairs.

18. The method of claim 17, wherein the method further comprises providing at least one site-specific nuclease.

19. The method of claim 18, wherein the site-specific nuclease is encoded by a portion of the nucleic acid template of the MiniVector.

20. The method of claim 18, wherein providing the site-specific nuclease comprises co-transfecting a separate MiniVector, a plasmid, a messenger RNA, or a virus encoding the site-specific nuclease, or a protein.

21. The method of claim 18, wherein the site-specific nuclease is selected from a group consisting of zinc finger nuclease (ZFN), transcription-activator-like effector nuclease (TALEN), meganuclease, and CRISPR (clustered regularly interspaced short palindromic repeats)/CAS (CRISPR associated) system.

22. The method of claim 18, wherein the site-specific nuclease induces one or more single stranded breaks in the target DNA sequence.

23. The method of claim 18, wherein the site-specific nuclease induces one or more double stranded breaks in the target DNA sequence.

24. The method of claim 17, wherein the alteration of the target DNA is mediated by a transposase or recombinase, including but not limited to the sleeping beauty transposon system.

25. The method of claim 17, wherein the MiniVector further comprises a chemical moiety, a modified oligonucleotide, and/or a modified backbone.

26. A method of treating a genetic disorder, or other condition, in a subject in need thereof, wherein an alteration of a target DNA sequence is desired, comprising:
a) administering to a subject a therapeutically effective amount of a MiniVector comprising a nucleic acid sequence template, wherein the nucleic acid sequence template comprises at least one portion complementary to a nucleic acid sequence near the target DNA sequence; and at least one portion which is not complementary to the target DNA sequence, wherein the non-complementary portion of the nucleic acid template contains the desired alteration;
b) base pairing of the complementary regions of the nucleic acid sequence template with the nucleic acid sequence near the target DNA sequence, with the exception of the non-complementary portion; and
c) incorporating the desired alteration into the targeted DNA sequence in a sequence-specific manner,
wherein the MiniVector lacks both a bacterial origin of replication and an antibiotic selection gene, and wherein the MiniVector has a size up to about 2,500 base pairs.

27. The method of claim 26, further comprising co-administering at least one site-specific nuclease.

28. The method of claim 26, wherein the site-specific nuclease is encoded by a portion of the nucleic acid template of the MiniVector.

29. The method of claim 27, wherein the co-administering comprises providing a separate MiniVector, a plasmid, a messenger RNA, or a virus encoding the site-specific nuclease, or a protein.

30. The method of claim 27, wherein the site-specific nuclease is selected from a group consisting of zinc finger nuclease (ZFN), transcription-activator-like effector nuclease (TALEN), meganuclease, and CRISPR (clustered regularly interspaced short palindromic repeats)/CAS (CRISPR associated) system.

31. The method of claim 27, wherein the site-specific nuclease induces one or more single stranded breaks in the target DNA sequence.

32. The method of claim 27, wherein the site-specific nuclease induces one or more double stranded breaks in the target DNA sequence.

33. The method of claim 26, wherein the alteration of the target DNA is mediated by a transposase or recombinase, including but not limited to sleeping beauty transposon system.

34. The method of claim 26, wherein the MiniVector further comprises a chemical moiety, a modified oligonucleotide, and/or a modified backbone.

35. The method of claim 26, wherein the subject is a mammal or a plant.


SEQUENCE LISTING (2 sequences)

SEQ ID NO: 1 - DNA, 50 bp, Homo Sapiens
actacacccagggaatgaagagcaagcgccatgttgaagccatcattacc

SEQ ID NO: 2 - DNA, 50 bp, Artificial Sequence
  Donor Template
actacacccagggaatctcgagcaagcgccatgttgaagccatcattacc